(19) United States
(12) Patent Application Publication
(10) Pub. No.: US 2025/0339025 A1
(43) Pub. Date: Nov. 6, 2025

(54) Compact Adaptive Optics Scanning Light Ophthalmoscope With Scalable Pupil

(71) Applicant: Wisconsin Alumni Research Foundation, Madison, WI (US)

(72) Inventor: Jeremy Rogers, Madison, WI (US)

(21) Appl. No.: 19/189,756

(22) Filed: Apr. 25, 2025

Related U.S. Application Data
(60) Provisional application No. 63/642,249, filed on May 3, 2024.

(51) Int. Cl. A61B 3/12; A61B 3/14

(52) U.S. Cl. CPC A61B 3/12; A61B 3/14

(57) ABSTRACT

An ophthalmoscope provides one or more eyepiece lenses that provide an adjustable entrance pupil allowing the entrance pupil of the ophthalmoscope to match the entrance pupil of the eye to increase the collected image information within the constraints of the eye pupil size, reducing scan times for given light energy.

### CROSS REFERENCE TO RELATED APPLICATION

This application claims the benefit of U.S. provisional patent application 63/642,249 filed May 3, 2024, and hereby incorporated by reference in its entirety.

### STATEMENT REGARDING FEDERALLY SPONSORED RESEARCH OR DEVELOPMENT

- -

### BACKGROUND OF THE INVENTION

The present invention relates to ophthalmoscopes for viewing the retina, and in particular, to an adaptive optics, scanning light, ophthalmoscope (AOSLO) offering reduced scan time.

Ophthalmoscopes are optical instruments allowing a clinician to view the back of the eye including the retina, optic nerve, and associated vasculature. While a simple ophthalmoscope may provide a basic light and lens that may be hand held by the clinician, more sophisticated instruments may make use of a scannable laser and confocal imaging to produce high contrast images distinguishing among different depths of tissue. Such instruments may also use adaptive optics to correct for aberrations of the eye itself such as may affect the obtained image and are termed adaptive optics scanning laser ophthalmoscopes (AOSLO).

Despite the clinical potential of AOSLO, such instruments have not achieved widespread acceptance. This is likely, in part, a result of the long scanning times necessary to acquire an image (on the order 45 minutes) and relatively small area of the acquired images.

### SUMMARY OF THE INVENTION

The present invention greatly improves the efficiency of data gathering of AOSLO-type ophthalmoscopes by matching the entrance pupil of the ophthalmoscope optics to the entrance pupil of the eye being imaged, the latter which may change significantly depending on the particular eye size or patient age. These improvements in data collection, resulting from more efficient transfer of the laser light energy into the eye and better utilization of the resolution limiting components of the ophthalmoscope, can dramatically reduce practical scan times, potentially to as little as five minutes.

In one specific embodiment, the invention provides an ophthalmoscope having a scanning laser producing a laser beam scannable over an area of in vivo retinal tissue of an eye and an image sensor for receiving a reflected laser beam from the in vivo retinal tissue over an area defined by an instrument aperture. At least one eyepiece lens is positionable in an optical path between the eye and instrument aperture providing selectable different instrument entrance pupils with respect to the instrument aperture so that the instrument entrance pupil may be matched to an entrance pupil of the eye defined by the eye lens and the iris.

It is thus a feature of at least one object of the invention to increase the rate at which useful image information can be collected from the eye with the given iris dilation or size, in terms of field-of-view and resolution. By matching the entrance pupil of the eyepiece to that of the eye, image information can be maximized and offsetting aberration or noise from a loss of collected light, reduced.

The ophthalmoscope may use a set of different lenses providing different instrument entrance pupils.

It is thus a feature of at least one object of the invention to provide a simple and robust implementation of a lens system that can produce different entrance pupils by using discrete different lenses thereby avoiding the complexity of zoom lens structures and the like.

Each of the different lenses may be associated with a different optical path and a lens may be positioned in the optical path between the eye and wavefront corrector by moving the eye between the different optical paths.

It is thus a feature of at least one embodiment of the invention to allow fixed lens placement eliminating the complexity of and potential error introduced by movable lenses.

The ophthalmoscope may include an electronic controller receiving a signal dependent on the instrument entrance pupil provided by the at least one eyepiece lens, the electronic controller communicating with a display to display an image of retinal tissue acquired by the image sensor together with a scale dependent on the signal.

It is thus a feature of at least one embodiment of the invention to provide automatic adjustment of image scaling when different lenses are used.

The ophthalmoscope may further include an input for receiving geometric information about the eye and the scale may also be dependent on the geometric information about the eye.

It is thus a feature of at least one embodiment of the invention to receive other eye characterizing information such as eye length for accurate scale rendering.

These particular objects and advantages may apply to only some embodiments falling within the claims and thus do not define the scope of the invention.

### BRIEF DESCRIPTION OF THE DRAWINGS

FIG. 1 is a block diagram of an adaptive optics scanning laser ophthalmoscope (AOSLO) suitable for use with the present invention and providing an eyepiece lens assembly and various other components communicating with a central controller;

FIG. 2 is a simplified elevational cross-section of an eye showing the structures of the lens iris and calculation of entrance pupil;

FIG. 3a is a simplified representation of a first example eyepiece lens assembly providing a narrow entrance pupil and high angle of refraction;

FIG. 3b is a figure similar to FIG. 3a showing a location of the entrance pupil of the eyepiece lens assembly positioned at the eye lens so that the narrow entrance pupil of the eyepiece lens assembly can be accommodated by a small iris size and hence small entrance pupil of the eye and further showing the larger field-of-view resulting from the high angle of refraction

FIG. 4a is a figure similar to FIG. 3a showing a second example eyepiece lens assembly providing a wider entrance pupil and lower angle of refraction;

FIG. 4b is a figure similar to FIG. 4a showing an eye having a larger iris opening that can accommodate the larger entrance pupil of the eyepiece lens assembly while yielding a smaller field-of-view resulting from the lower angle of refraction.

FIG. 5 is a perspective view of the AOSLO of FIG. 1 employing multiple discrete eyepiece lenses on different optical paths selected by an internal mirror, and showing positioning of a patient support at different locations to select among the different lenses and different instrument exit apertures; and

FIG. 6 is a flowchart of a program executed by the central controller of FIG. 1 providing automatic scale calculation.

### DETAILED DESCRIPTION OF THE PREFERRED EMBODIMENT

Referring now to FIG. 1, an AOSLO ophthalmoscope 10 may use a laser 12 providing an output laser beam 16 received by an eye 20 being measured to reflect off the eye retina as a reflected laser beam 18. Generally the reflected laser beam 18 will be collinear with the output laser beam 16 and are shown separated only for clarity.

The output laser beam 16 and reflected laser beam 18 pass through an imaging and control optical assembly 14 including one or more beam splitters for separating the output laser beam 16 and reflected laser beam 18 so that the latter may pass to an image sensor such as a photomultiplier tube and a wavefront sensor (for example, a Shack Hartmann wavefront sensor) for detecting wavefront aberration for the purpose of compensating the same using a wavefront corrector. In addition, the imaging and control optical assembly 14 may include a pinhole mask for confocal operation as well as focus adjustment mechanisms. A n example imaging and control optical assembly 14 suitable for the present invention is described, for example, in N. Sredar, M. Razeen, B. Kowalski, J. Carroll, and A. Dubra, “Comparison of confocal and non-confocal split-detection cone photoreceptor imaging,” Biomed, Opt. Express, 12, 737-755 (2021) hereby incorporated by reference.

The laser beams 16 and 18 will be steered in a raster pattern across the retina of the eye 20 by means of a resonant scanner 22 providing a first Cartesian axis (e.g. horizontal) of angular redirection followed by a second piezoelectric scanner 24 providing the second Cartesian axis (e.g. vertical) of angular redirection. A wavefront corrector 26 may receive the raster scanned focal point of the laser 12 over an operative area of the wavefront corrector 26 by which wavefront correction may be implemented. Such wavefront correctors 26, for example, may provide for a mirrored surface that can be perturbed by underlying MEMS (micro-electromechanical systems) actuators. A wavefront corrector 26 suitable for the present invention may be a deformable mirror commercially available from ALPAO of France. Ideally, the raster scan area matches the active area of the wavefront corrector 26 both of which define an effective image aperture of the ophthalmoscope 10. Prior to receipt by the eye 20, the laser beams 16 and 18 are focused by an eyepiece lens assembly 30. Henceforth the eyepiece lens assembly 30 will be depicted as a simple single lens but it will be understood that it may generally include multiple lenses and mirrors either in fixed position or movable, for example, in the manner of a telephoto lens system.

Each of the components shown in FIG. 1 of the imaging and control optical assembly 14, the resonant scanner 22, the piezoelectric scanner 24, and the eyepiece lens assembly 30 may communicate with a controller 25, for example, having one or more processors 27 executing a stored program 29 stored in computer memory 31. The controller 25 may receive a signal from the eyepiece lens assembly 30 indicating particular optical properties of a selected eyepiece lens assembly 30, and may provide signals controlling the wavefront corrector 26 and the resonant scanner 22 and piezoelectric scanner 24 for the purpose of scanning the eye 20 as is generally understood in the art.

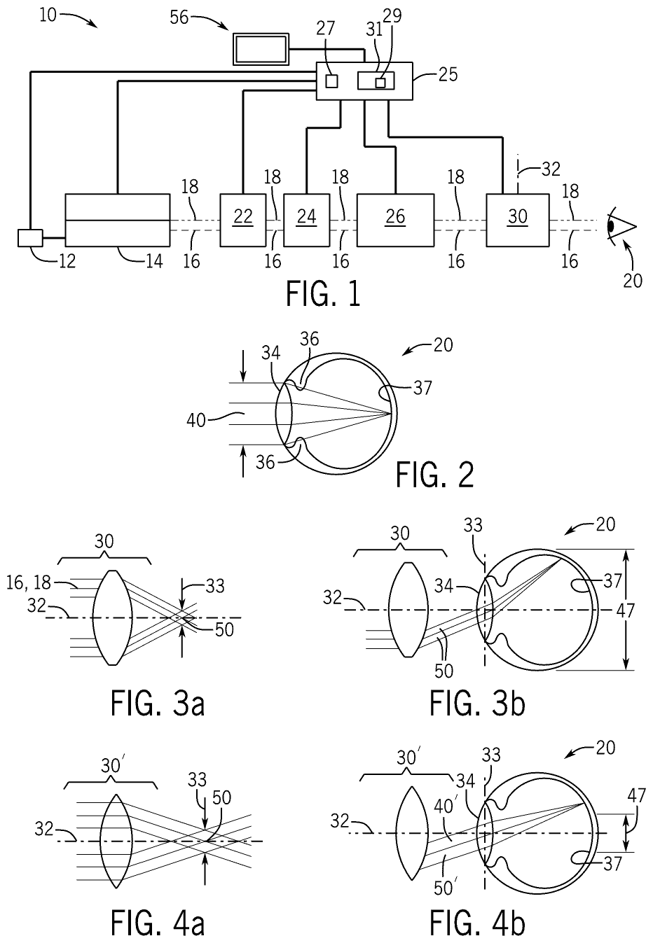

Referring now also to FIG. 2, the eye 20 provides a cornea including a lens 34 and iris 36 that together define an entrance pupil 40 of the eye 20. Generally, the entrance pupil 40 will reflect the size of a beam of light with parallel rays that can be received through the lens 34 and iris 36 to converge on a point on the retina 37. This entrance pupil 40 is relatively constant with distance from the lens 34 in front of the eye 20 but will be generally measured proximate to the lens 34. The entrance pupil 40 generally differs from the size of the iris 36 because of the interposition of the lens 34 whose refractive properties change the apparent size of the iris 36.

Referring now to FIG. 3a, in a similar fashion, the eyepiece lens assembly 30 also provides an entrance pupil 50 being the size of the laser beam 16 passing out of the eyepiece lens assembly 30 toward the eye 20. This size will be a function of the effective refractive power of the eyepiece lens assembly 30 with higher refractive powers producing smaller entrance pupils 50.

The position of the laser beam 16 will be scanned over an area of the eyepiece lens assembly 30 by the resonant scanner 22 and piezoelectric scanner 24 to be refracted by the eyepiece lens assembly 30 toward the optical axis 32 of the eyepiece lens assembly 30. A point at which the centers of the beams converge define a convergence plane 33 at a distance in front of the eyepiece lens assembly 30 again being a function of its focal length or refractive power.

In comparison and referring now to FIG. 4a, a second eyepiece lens assembly 30′ having a longer focal length and lower refractive power will produce a larger entrance pupil 50′ with a convergence plane 33′ at a greater distance in front of the eyepiece lens assembly 30.

Referring now to FIGS. 3b and 4b, depicting respectively the eyepiece lens assemblies 30 and 30′ of FIGS. 3a and 4a, the eyepiece lens assemblies 30 and 30′ may be selected to match eyes 20 and 20′ whose entrance pupils 40 and 40′ best match the corresponding entrance pupils 50 and 50′ of eyepiece lens assemblies 30 and 30′. That is, the smaller entrance pupil 50 of eyepiece lens assembly 30 may be used for eye 20 having a smaller iris size and hence smaller entrance pupil 40 while the larger entrance pupil 50′ of eyepiece lens assembly 30′ may be used for eye 20′ having a larger entrance pupil 40′. The respective convergence planes 33 of the respective eyepiece lens assemblies 30 and 30′ are positioned at the lens 34.

Generally, the eyepiece lens assembly 30 having a smaller entrance pupil 50 would be expected to produce lower resolution images and thus to be disfavored over the optical lens assembly 30′ having a larger entrance pupil 50′ supporting higher resolution. The present inventor, however, has recognized that this resolution disadvantage is offset by a larger field of view 47 obtained with the eyepiece lens assembly 30 such as provides additional data collection favorably affecting scan time. The field-of-view 47 is the range of illuminated points on the retina 37 possible for a given scan angulation of the resonant scanner 22 and piezoelectric scanner 24. In addition, the optical lens assembly 30 provides better light coupling into the eye providing an improved signal-to-noise ratio in the acquired image data. Both eyepiece lens assemblies exploit the full range of the wavefront corrector 26 and resonant scanner 22 and piezoelectric scanner 24 making them equal as far as utilizing these other components of the ophthalmoscope 10.

Generally it is contemplated that multiple different eyepiece lens assemblies 30 will be employed each having a different entrance aperture 50 suitable for different patients or eyes and generally matching the expected entrance apertures 40 of those eyes.

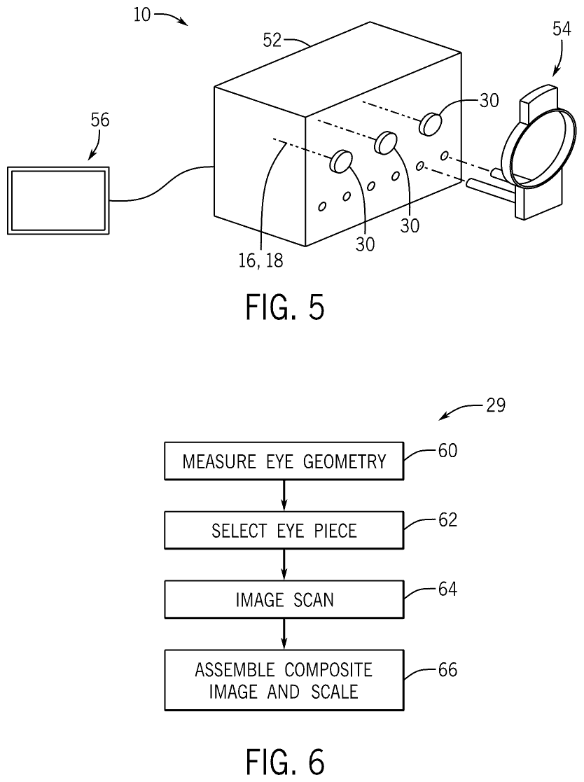

Referring now to FIG. 5, the necessary different instrument entrance pupils 50 can be obtained by substituting lenses having different refractive properties but may preferably be performed by providing multiple eyepiece lens assemblies 30, for example, directed outwardly on a housing 52 of the ophthalmoscope 10 and having predetermined fixed and calibrated positions that can be precisely maintain. A particular eyepiece lens assembly 30 may be selected by an internal folding mirror directing the laser beams 16 and 18 along optical paths to the different eyepiece lens assemblies 30. A head support 54, for example, providing for a head and chin rest to locate a patient with respect to the housing 52 and the eyepiece lens assemblies 30 may be selectively engaged in front of different of the eyepiece lens assemblies 30 according to the eyepiece lens assembly 30 selected. The head support 54 may provide an engagement depth when inserted into the housing 52 set to match the desired lens eye relief.

A user terminal 56 may communicate with the ophthalmoscope 10 and particularly with the controller 25 to provide information, for example, obtained from a separate device that can measure the iris size (for the purpose of selecting an eyepiece lens assembly 30) and eye geometry, for example, eyeball length. This eyeball length and knowledge of the selected eyepiece lens assembly 30 may be used to establish a scale of a resulting image collected from the image sensor of the imaging and control optical assembly 14, the scale, for example being a ruler like indicia on the image.

Referring now generally to FIG. 6, the program 29 executed on the controller 25 may operate, for example, to receive eye measurements including iris diameter and eyeball length per process block 60, this information, for example, being obtained from a biometer of a type commercially available from Zeiss Meditec, Dublin CA under the trade name 10L M aster.

At process block 62, information from the biometer or obtained directly from the ophthalmoscope 10, for example, by measuring light throughput, may be used to select a particular eyepiece lens assembly 30 or to adjust a multicomponent zoom type lens to provide the entrance pupil matching discussed above.

At process blocks 64, an image scan is conducted of multiple small regions (to minimize effective eye-movement) with those regions incrementally tiled across the retina 37. At process block 66, the individual regions of the scan assembled, for example, by matching overlapping portions to provide an image of a larger area of the retina with a scale provided on the image indicating its absolute size deduced from the information about eyeball length and selected eyepiece lens assembly 30.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context. The term “entrance pupil” should be understood as defined herein and may in some cases be equivalent to a measurement termed “exit pupil”.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. A II of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An ophthalmoscope comprising:
a scanning laser producing a laser beam scannable over an area of in vivo retinal tissue of an eye;
an image sensor for receiving a reflected laser beam from the in vivo retinal tissue over an area defined by an instrument aperture;
at least one eyepiece lens positionable in an optical path between the eye and instrument aperture providing selectable different instrument entrance pupils with respect to the instrument aperture,
whereby the instrument entrance pupil may be matched to an entrance pupil of the eye defined by the eye lens and the eye iris.

2. The ophthalmoscope of claim 1 wherein the at least one eyepiece lens is a set of different lenses providing different instrument entrance pupils.

3. The ophthalmoscope of claim 2 wherein each of the different lenses is associated with a different optical path and wherein a lens is positioned in the optical path between the eye and wavefront corrector by moving the eye between the different optical paths.

4. The ophthalmoscope of claim 1 wherein the scanning laser includes a laser providing a collimated beam and a raster scanning actuator system for deflecting the beam in a raster pattern over an area commensurate with the instrument aperture.

5. The ophthalmoscope of claim 1 further including a wavefront corrector positioned in an optical path between scanning laser and the image sensor and providing an active area of wavefront correction commensurate with the instrument aperture.

6. The ophthalmoscope of claim 1 further including an electronic controller receiving a signal dependent on the instrument entrance pupil provided by the at least one eyepiece lens, the electronic controller communicating with a display to display an image of retinal tissue acquired by the image sensor together with a scale dependent on the signal.

7. The ophthalmoscope of claim 6 further including an input for receiving geometric information about the eye and wherein the scale is also dependent on the geometric information about the eye.
